(19) United States
(12) Patent Application Publication
(10) Pub. No.: US 2009/0215077 A1
(43) Pub. Date: Aug. 27, 2009

(54) METHODS FOR DIAGNOSIS OF ACUTE CORONARY SYNDROME

(75) Inventors: Mark B. CHANDLER, Austin, TX (US); Michael D. SPAIN, Austin, TX (US); James MAPES, Austin, TX (US); George RODGERS, Austin, TX (US)

Correspondence Address: FOLEY AND LARDNER LLP;SUITE 500, 3000 K STREET NW, WASHINGTON, DC, 20007, US

(73) Assignee: Rules-Based Medicine, Inc.

(21) Appl. No.: 12/417,496

(22) Filed: Apr. 2, 2009

Related U.S. Application Data
(63) Continuation of application No. 11/475,249, filed on Jun. 27, 2006.
(60) Provisional application No. 60/694,666, filed on Jun. 29, 2005.

(51) Int. Cl. G01N 33/53; C12Q 1/00; C12Q 1/48

(52) U.S. Cl. 435/7.1; 435/4; 435/15

(57) ABSTRACT

Provided are methods for the detection and diagnosis of acute coronary syndrome or ACS. The methods are based on the discovery that abnormal levels of selected analytes in sample fluid, typically blood samples, of patients who are at risk are supportive of a diagnosis of ACS. At least two new biomarkers for ACS are thus disclosed, MMP-3 and SGOT. Altogether the concentrations of twelve analytes provide a sensitive and selective picture of the patient's condition, namely, whether the patient is suffering a heart attack. Other important biomarkers for ACS are described, including but not limited to IL-18, Factor VII, ICAM-1, Creatine Kinase-MB, MCP-1, Myoglobin, C Reactive Protein, von Willebrand Factor, TIMP-1, Ferritin, Glutathione S-Transferase, Prostate Specific Antigen (free), IL-3, Tissue Factor, alpha-Fetoprotein, Prostatic Acid Phosphatase, Stem Cell Factor, MIP-1-beta, Carcinoembryonic Antigen, IL-13, TNF-alpha, IgE, Fatty Acid Binding Protein, ENA-78, IL-1-beta, Brain-Derived Nerotrophic Factor, Apolipoprotein A1, Serum Amyloid P, Growth Hormone, Beta-2 microglobulin, Lipoprotein (a), MMP-9, Thyroid Stimulating hormone, alpha-2 Macroglobulin, Complement 3, IL-7, Leptin, and IL-6. Kits containing reagents to assist in the analysis of fluid samples are also described.

### CROSS REFERENCE TO RELATED APPLICATIONS

This application is a continuation of U.S. patent application Ser. No. 11/475,249, filed Jun. 27, 2006, which claims priority to U.S. Provisional Patent Application No. 60/694,666, filed Jun. 29, 2005, the entire disclosures of which are incorporated herein by reference.

### BACKGROUND

1. Field of the Invention

Methods, kits and reagents for detection and/or diagnosis of acute coronary syndrome (ACS).

2. Description of the Related Art

Cardiovascular disease is the number one killer in the United States. Most victims succumb to a massive heart attack and never make it to the emergency room. The lucky ones might complain of chest pain, headache, dizziness and are rushed to the hospital. However, not all patients suffering from acute coronary syndrome (ACS) are properly diagnosed. Every year several thousand heart attack patients who reported to a hospital were sent home. In the U.S., out of 8,000,000 people reporting chest pain, 3,000,000 are sent home as being non-cardiac in nature, but 40,000 of these suffer myocardial infarction. Of 5,000,000 kept in hospital for suspected cardiac etiology, 2,500,000 (50%) are non-cardiac, 1,000,000 suffer MI, 1,2000,000 suffer unstable angina, and 300,000 cardiac arrest. These statistics provide the primary rationale for improving the screening and diagnosis of patients at risk for ACS.

The selectivity and sensitivity of current assays for ACS are lacking, with the frequency of false positives and false negatives at an undesirable level. Thus, there is a critical need to develop additional biomarkers for early detection of cardiovascular disease and, in particular, for the accurate diagnosis of heart attacks.

### SUMMARY OF THE INVENTION

A method for rapid detection and/or accurate diagnosis of ACS is provided. The method can be practiced with a determination of the concentrations of one or two biomarkers in a patient fluid sample. Elevated (or depressed, as the case might be) levels of the one or two biomarkers, which are statistically different from levels found in “normals” (that is, control subjects not suffering from ACS), support a positive diagnosis of ACS. Preferably, the method utilizes a panel of analytes or “biomarkers,” up to twelve or more substances found in a sample fluid (e.g., whole blood, serum, plasma, or urine), to help support a positive or negative diagnosis of ACS. Up to 99% accuracy in making a correct diagnosis is provided by the method.

According to the invention a method of diagnosing acute coronary syndrome (ACS) in a human subject suspected of suffering from ACS is provided, which comprises: (a) obtaining a fluid sample from a human subject suspected of suffering from ACS; (b) determining the concentration of MMP-3 in said fluid sample; (c) deciding if the determined concentration of MMP-3 in said fluid sample is statistically different from that found in a control group of human subjects, whereby a statistically different elevated concentration of MMP-3 supports a positive diagnosis of ACS. Typically, the human subject is complaining of chest pains. Any one of a number of fluid samples can be tested. Preferably, the fluid sample is selected from whole blood, plasma, serum, or urine. It has been discovered that a measured concentration of about 1 ng/mL or above of MMP-3 in the fluid sample supports a positive diagnosis of ACS.

In another aspect of the invention a method is provided for diagnosing acute coronary syndrome (ACS) in a human subject suspected of suffering from ACS, which method comprises: (a) obtaining a fluid sample from a human subject suspected of suffering from ACS; (b) determining the concentration of SGOT in said fluid sample; (c) deciding if the determined concentration of SGOT in said fluid sample is statistically different from that found in a control group of human subjects, whereby a statistically different depressed concentration of SGOT supports a positive diagnosis of ACS. It has been found that a measured concentration of about 10 μg/mL or below of SGOT in said fluid sample supports a positive diagnosis of ACS.

Still another aspect of the invention relates to a method of diagnosing acute coronary syndrome (ACS) in a human subject suspected of suffering from ACS, comprising: (a) obtaining a fluid sample from a human subject suspected of suffering from ACS; (b) determining the concentrations of MMP-3 and SGOT in said fluid sample; (c) deciding if the determined concentrations of MMP-3 and SGOT in said fluid sample are statistically different from that found in a control group of human subjects, whereby a statistically different elevated concentration of MMP-3 and a statistically different depressed concentration of SGOT together support a positive diagnosis of ACS.

In a preferred embodiment, the method of the invention further comprises determining the concentration in said fluid sample of at least one of IL-18, Factor VII, ICAM-1, Creatine Kinase-MB, MCP-1, Myoglobin, C Reactive Protein, TIMP-1, Ferritin, or Glutathione S-Transferase, or any combination thereof. It has been found that statistically different elevated concentrations, compared to control levels, of all analytes mentioned above except SGOT, support a positive diagnosis of ACS. In particular, certain threshold levels of analytes in the sample fluids have been found to be important in the detection or diagnosis of ACS, including IL-18 (about 300 pg/mL or above), Factor VII (about 320 ng/mL or above), ICAM-1 (about 170 ng/mL or above), Creatine Kinase-MB (about 5 ng/mL or above), MCP-1 (about 275 pg/mL or above), Myoglobin (about 30 ng/mL or above), C Reactive Protein (about 11 μg/mL or above), TIMP-1 (about 120 ng/mL or above), Ferritin (about 300 ng/mL or above), and Glutathione S-Transferase (about 2 ng/mL or above.

Yet other biomarkers have been determined to be useful in arriving at a positive or negative diagnosis of ACS. These biomarkers include, in addition to those already disclosed, Prostate Specific Antigen (free), IL-3, Tissue Factor, alpha-Fetoprotein, Prostatic Acid Phosphatase, Stem Cell Factor, MIP-1-beta, Carcinoembryonic Antigen, IL-13, TNF-alpha, IgE, Fatty Acid Binding Protein, ENA-78, IL-1-beta, Brain-Derived Nerotrophic Factor, Apolipoprotein A1, Serum Amyloid P, Growth Hormone, Beta-2 microglobulin, Lipoprotein (a), MMP-9, Thyroid Stimulating hormone, alpha-2 Macroglobulin, Complement 3, IL-7, Leptin, and IL-6.

Various techniques for assessing the importance of certain biomarkers in arriving at a diagnosis is also described herein. One such technique is a projection of compiled results on a proximity map, whereby the proximity of a subject's determined concentrations to a cluster of other subjects' determined concentrations, who were previously diagnosed as having suffered from ACS, contributes to a positive diagnosis of ACS. Other techniques include the application of one or more statistical methods (e.g., linear regression analysis, classification tree analysis, heuristic nave Bayes analysis and the like).

Also provided is a kit comprising reagents for determining the concentration in a fluid sample of a panel of analytes including MMP-3, SGOT and one or more of IL-18, Factor VII, ICAM-1, Creatine Kinase-MB, MCP-1, Myoglobin, C Reactive Protein, TIMP-1, Ferritin, or Glutathione S-Transferase. The reagents may include antibodies against the members of a given panel of analytes. Furthermore, the reagent may be immobilized on a substrate, which substrate may comprise a two-dimensional array, a microtiter plate, or multiple bead sets.

The methods may further comprise comparing the levels of the one, two, or more biomarkers in a patient's blood with levels of the same biomarkers in one or more control samples by applying a statistical method such as: linear regression analysis, classification tree analysis and heuristic nave Bayes analysis. The statistical method may be, and typically is performed by a computer process, such as by commercially available statistical analysis software. In one embodiment, the statistical method is a classification tree analysis, for example CART (Classification and Regression Tree). Results for a particular patient or subject, whose sample fluid is tested against a panel of biomarkers according to the method, can be projected onto a proximity map. The proximity of a particular patient's biomarker concentration results to one of at least two populations (those previously diagnosed as having suffered a heart attack and normals) supports a either a positive or negative diagnosis of ACS.

An article of manufacture is provided which comprises binding reagents specific for at least one of MMP-3 and SGOT, preferably both biomarkers. More preferably, a kit is provided which comprises binding reagents specific for MMP-3, SGOT, IL-18, Factor VII, ICAM-1, Creatine Kinase-MB, MCP-1, Myoglobin, C Reactive Protein, TIMP-1, Ferritin, or Glutathione S-Transferase. In a preferred embodiment, each binding reagent is immobilized on a substrate. For example, monoclonal antibodies against MMP-3, SGOT and the other biomarkers described herein are immobilized independently to one or more discrete locations on one or more surfaces of one or more substrates. The substrates may be beads comprising an identifiable biomarker, wherein each binding reagent is attached to a bead comprising a different identifiable biomarker than beads to which a different binding reagent is attached. The identifiable biomarker may comprise a fluorescent compound, a quantum dot, or the like.

In another embodiment, a method is provided for determining the occurrence of a heart attack in a patient, comprising determining levels of at least one of MMP-3 and SGOT.

In a further embodiment, a method of predicting onset of cardiovascular disease is provided, comprising determining the change in concentration at two or more points in time of two or more markers in a patient's blood, wherein an observed increase in the concentration of MMP-3, a decrease in the concentration of SGOT or both, in the patient's blood between the two time points, is predictive of the onset of cardiovascular disease.

Other aspects of the invention will become apparent to those of ordinary skill after considering the detailed descriptions provided herewith.

### BRIEF DESCRIPTION OF THE DRAWINGS

FIG. 1 is a projection of a proximity map of patients whose fluid samples were tested against a panel of biomarkers listed on the right-hand margin. The results of this proximity map analysis indicate that consideration of all the biomarkers listed provides a degree of accuracy of a correct diagnosis of ACS of about 98%, with subjects having suffered a heart attack (or ACS) positioned on the left-hand side of the figure (red or light gray dots) and subjects who have not suffered a heart attack positioned on the right-hand side of the figure (blue or dark gray spots).

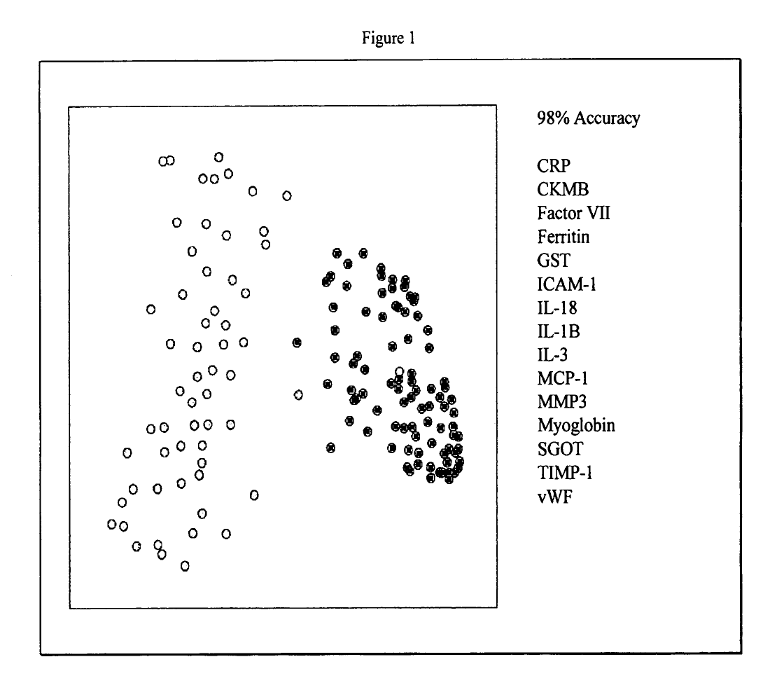

FIG. 2 is a projection of a proximity map of patients whose fluid samples were tested against a panel of biomarkers listed on the right-hand margin. The results of this proximity map analysis indicate that consideration of all the biomarkers listed provides a degree of accuracy of a correct diagnosis of ACS of about 97%, with subjects having suffered a heart attack (or ACS) positioned on the left-hand side of the figure (red or light gray dots) and subjects who have not suffered a heart attack positioned on the right-hand side of the figure (blue or dark gray spots).

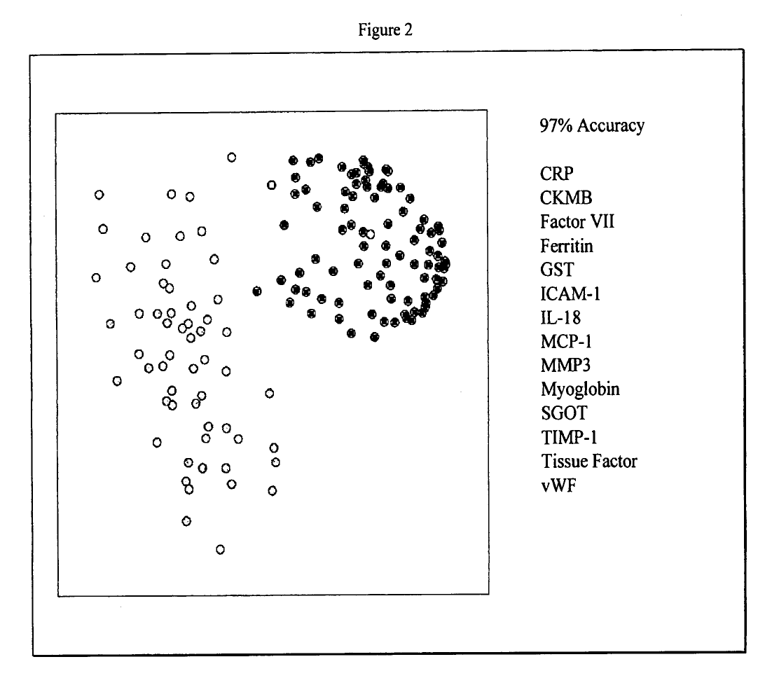

FIG. 3 is a projection of a proximity map of patients whose fluid samples were tested against a panel of biomarkers listed on the right-hand margin (except that the results for the biomarkers Tissue Factor and vWF were excluded from the analysis). The results of this proximity map analysis indicate that consideration of all the biomarkers listed provides a degree of accuracy of a correct diagnosis of ACS of about 99%, with subjects having suffered a heart attack (or ACS) positioned on the left-hand side of the figure (red or light gray dots) and subjects who have not suffered a heart attack positioned on the right-hand side of the figure (blue or dark gray spots).

FIG. 4 is a projection of a proximity map of patients whose fluid samples were tested against a panel of biomarkers listed on the right-hand margin. The results of this proximity map analysis indicate that consideration of all the biomarkers listed provides a degree of accuracy of a correct diagnosis of ACS of about 94%, with subjects having suffered a heart attack (or ACS) positioned on the left-hand side of the figure (red or light gray dots) and subjects who have not suffered a heart attack positioned on the right-hand side of the figure (blue or dark gray spots).

FIG. 5 is a projection of a proximity map of patients whose fluid samples were tested against a panel of biomarkers listed on the right-hand margin. The results of this proximity map analysis indicate that consideration of all the biomarkers listed provides a degree of accuracy of a correct diagnosis of ACS of about 97%, with subjects having suffered a heart attack (or ACS) positioned on the left-hand side of the figure (red or light gray dots) and subjects who have not suffered a heart attack positioned on the right-hand side of the figure (blue or dark gray spots).

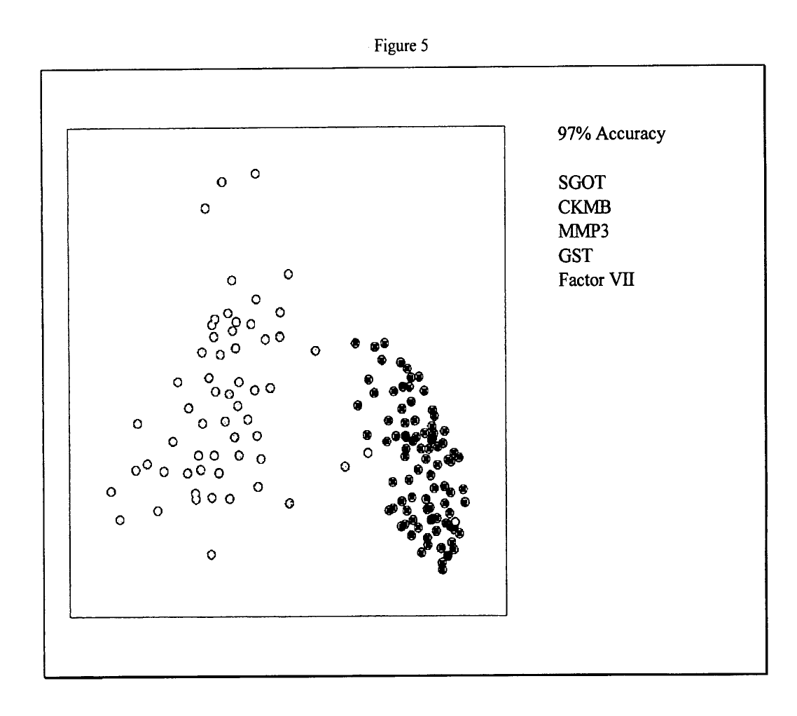

### DETAILED DESCRIPTION OF THE PREFERRED EMBODIMENTS

The use of numerical values in the various ranges specified in this application, unless expressly indicated otherwise, are stated as approximations as though the minimum and maximum values within the stated ranges were both preceded by the word “about.” In this manner, slight variations above and below the stated ranges can be used to achieve substantially the same results as values within the ranges. Also, the disclosure of these ranges is intended as a continuous range including every value between the minimum and maximum values.

Provided herein is a multifactorial assay for rapid identification of a heart attack patient. Identified below are certain sample fluid (e.g., blood) analytes or biomarkers useful in the detection and/or diagnosis of ACS. It has been found that the following biomarkers are over-expressed in the blood of patients suffering from or who have suffered a heart attack. SGOT has been found to be under-expressed in patients suffering from or who have suffered a heart attack.

Also identified as being useful in the detection or proper diagnosis of subjects suffering from or who have suffered a heart attack are the biomarkers MMP-3, SGOT, IL-18, Factor VII, ICAM-1, Creatine Kinase-MB, MCP-1, Myoglobin, C Reactive Protein, von Willebrand Factor, TIMP-1, Ferritin, Glutathione S-Transferase, Prostate Specific Antigen (free), IL-3, Tissue Factor, alpha-Fetoprotein, Prostatic Acid Phosphatase, Stem Cell Factor, MIP-1-beta, Carcinoembryonic Antigen, IL-13, TNF-alpha, IgE, Fatty Acid Binding Protein, ENA-78, IL-1-beta, Brain-Derived Nerotrophic Factor, Apolipoprotein A1, Serum Amyloid P, Growth Hormone, Beta-2 microglobulin, Lipoprotein (a), MMP-9, Thyroid Stimulating hormone, alpha-2 Macroglobulin, Complement 3, IL-7, Leptin, or IL-6.

The parameters for establishing the significance of one or more biomarkers for the diagnosis of ACS are determined statistically by comparing normal or control blood (preferably, e.g., serum or plasma) levels of these biomarkers with blood levels in patients clinically and properly diagnosed as having suffered from or is having a heart attack. The statistical data presented below in Table 1 identify certain mean values and accompanying standard deviations for the blood levels of the above-described biomarkers in heart attack patients and in normals. As a non-limiting example of estimates of significant threshold values in support of a positive diagnosis of ACS, the following concentrations are provided: MMP-3 (about 1 ng/mL or above), SGOT (about 10 μg/mL or below), IL-18 (about 300 pg/mL or above), Factor VII (about 320 ng/mL or above), ICAM-1 (about 170 ng/mL or above), Creatine Kinase-MB (about 5 ng/mL or above), MCP-1 (about 275 pg/mL or above), Myoglobin (about 30 ng/mL or above), C Reactive Protein (about 11 μg/mL or above), TIMP-1 (about 120 ng/mL or above), Ferritin (about 300 ng/mL or above), or Glutathione S-Transferase (about 2 ng/mL or above).

It is understood that these values are approximate. Statistical methods can be used to define the critical range of values. Typically within one standard deviation of those approximate values might be considered as statistically significant values for determining a statistically significant difference, preferably two standard deviations. For this reason, the word “about” is used in connection with the stated values. “Statistical classification methods” are used to identify biomarkers capable of discriminating normal patients from patients with ACS and are further used to determine critical blood values for each biomarker for discriminating between such patients. Certain statistical methods can be used to identify discriminating biomarkers and panels thereof. These statistical methods may include, but are not limited to: 1) linear regression; 2) classification tree methods; and 3) statistical machine learning to optimize the unbiased performance of algorithms for making predictions. Each of these statistical methods is well-known to those of ordinary skill in the field of biostatistics and can be performed as a process in a computer. A large number of software products are available commercially to implement statistical methods, such as, without limitation, S-PLUS™, commercially available from Insightful Corporation of Seattle, Wash.

By identifying biomarkers useful in the determination and/or diagnosis of ACS and by use of statistical methods to identify which biomarkers and groups of biomarkers are particularly useful in identifying ACS-at-risk patients, a person of ordinary skill in the art, based on the disclosure herein, can compose panels of biomarkers having superior selectivity and sensitivity. Examples of biomarkers that can be included in panels, which provide excellent discriminatory capability, include: MMP-3, SGOT, IL-18, Factor VII, ICAM-1, Creatine Kinase-MB, MCP-1, Myoglobin, C Reactive Protein, von Willebrand Factor, TIMP-1, Ferritin, Glutathione S-Transferase, Prostate Specific Antigen (free), IL-3, Tissue Factor, alpha-Fetoprotein, Prostatic Acid Phosphatase, Stem Cell Factor, MIP-1-beta, Carcinoembryonic Antigen, IL-13, TNF-alpha, IgE, Fatty Acid Binding Protein, ENA-78, IL-1-beta, Brain-Derived Nerotrophic Factor, Apolipoprotein A1, Serum Amyloid P, Growth Hormone, Beta-2 microglobulin, Lipoprotein (a), MMP-9, Thyroid Stimulating hormone, alpha-2 Macroglobulin, Complement 3, IL-7, Leptin, and IL-6. Examples of specific panels comprising selected biomarkers from the above-mentioned list, include, but are not limited to: (i) CRP, CKMB, Factor VII, Ferritin, GST, ICAM-1, IL-18, IL-1B, IL-3, MCP-1, MMP-3, Myoglobin, SGOT, TIMP-1 and vWF; (ii) CRP, CKMB, Factor VII, Ferritin, GST, ICAM-1, IL-18, MCP-1, MMP-3, Myoglobin, SGOT, TIMP-1, Tissue Factor and vWF; (iii) CRP, CKMB, Factor VII, Ferritin, GST, ICAM-1, IL-18, MCP-1, MMP-3, Myoglobin, SGOT and TIMP-1; (iv) SGOT, CKMB, MMP-3, GST, Factor VII, IL-18, IL-3, MCP-1, ICAM-1 and IL-1B; (v) SGOT, CKMB, MMP-3, GST and Factor VII; (vi) SGOT and MMP-3. It will be recognized by those of ordinary skill in the field of biostatistics, that the number of biomarkers in any given panel may be different depending on the combination of biomarkers. With optimum sensitivity and specificity being the goal, one panel may include two biomarkers, another may include five, and still others may include twelve or more, yielding similar results.

The invention is based on an evaluation of at least MMP-3 levels, alone or in combination with levels of immunological SGOT and/or other biomarkers, in serum for diagnosis of acute coronary syndromes (unstable angina, acute myocardial infarction, sudden cardiac death, coronary plaque rupture, or thrombosis) in all stages of their occurrence. The invention is also based on the evaluation of at least immunological SGOT levels, optionally in combination with levels of at least MMP-3. Patients with acute coronary syndromes are at considerable risk for death and serious complications, and outcomes can be improved with appropriate diagnosis and therapy. Thus, rapid and accurate diagnosis of patients complaining of chest pain is critical for patient care.

The results described herein demonstrate that serum MMP-3 levels are elevated in unstable angina and acute myocardial infarction. Thus, MMP-3 can be used as an early biomarker of inflammatory cardiac conditions, and in particular, acute coronary syndrome. By the same token, SGOT levels are depressed in unstable angina and acute myocardial infarction. Thus, SGOT can be used as an early biomarker of acute coronary syndrome.

The present method includes measuring the level of MMP-3 and/or SGOT in a biological sample (e.g., whole blood, plasma, serum or urine and the like) from a patient; comparing the respective levels with that of control subjects; and diagnosing the state of disease based on the level of MMP-3 or SGOT relative to that of control subjects. A patient can be diagnosed with ACS if the level of MMP-3 is increased relative to that of control subjects or if SGOT is decreased relative to controls.

A typical control value for MMP-3 is in the range of about 0.1-0.8 ng/mL. A concentration of about 1 ng/mL or above in a patient sample supports a positive diagnosis. The general range for elevated values of MMP-3 is about 1.5-20 ng/mL.

A typical control value for SGOT is in the range of about 17-25 μg/mL. An immunological concentration of about 10 μg/mL or below in a patient sample supports a positive diagnosis. SGOT is often measured enzymatically. However, here we are measuring the amount of protein which is present, which may include enzymatically inactive plus enzymatically active SGOT. The general range for depressed values of immunological SGOT concentration is about 15-1 μg/mL.

MMP-3 and SGOT can be captured with anti-MMP-3 and anti-SGOT polyclonal antibodies, respectively, or with corresponding monoclonal antibodies. The diagnostic method may also include measuring the levels of one or more additional analytes selected from the group consisting of: IL-18, Factor VII, ICAM-1, Creatine Kinase-MB, MCP-1, Myoglobin, C Reactive Protein, von Willebrand Factor, TIMP-1, Ferritin, Glutathione S-Transferase, Prostate Specific Antigen (free), IL-3, Tissue Factor, alpha-Fetoprotein, Prostatic Acid Phosphatase, Stem Cell Factor, MIP-1-beta, Carcinoembryonic Antigen, IL-13, TNF-alpha, IgE, Fatty Acid Binding Protein, ENA-78, IL-1-beta, Brain-Derived Nerotrophic Factor, Apolipoprotein A1, Serum Amyloid P, Growth Hormone, Beta-2 microglobulin, Lipoprotein (a), MMP-9, Thyroid Stimulating hormone, alpha-2 Macroglobulin, Complement 3, IL-7, Leptin, and IL-6; and diagnosing the patient's condition based on the level of the additional analyte and the level of MMP-3 and/or SGOT relative to that of control subjects.

TABLE 1
Significant Analytes in ACS Study

| | | ACS | ACS | Control | Control | P value |
|---|---|---|---|---|---|---|
| | Units | Mean | SD | Mean | SD | (t-test) |
| IL-18 | pg/mL | 378 | 184 | 128 | 88 | 8.7E−22 |
| Factor VII | ng/mL | 523 | 170 | 205 | 58 | 1.7E−21 |
| SGOT | ug/mL | 5.7 | 9.8 | 21 | 4.4 | 6.3E−21 |
| ICAM-1 | ng/mL | 216 | 92 | 108 | 31 | 2.0E−19 |
| Creatine Kinase-MB | ng/mL | 32 | 25 | 0.69 | 0.70 | 2.0E−19 |
| MCP-1 | pg/mL | 349 | 159 | 151 | 124 | 9.4E−15 |
| Myoglobin | ng/mL | 73 | 71 | 16 | 7.8 | 2.8E−13 |
| MMP-3 | ng/mL | 10 | 8.5 | 0.41 | 0.31 | 4.9E−11 |
| C Reactive Protein | ug/mL | 17 | 19 | 3.4 | 4.1 | 9.5E−11 |
| von Willebrand Factor | ug/mL | 25 | 14 | 12 | 9.4 | 2.2E−10 |
| TIMP-1 | ng/mL | 136 | 50 | 100 | 18 | 1.6E−09 |
| Ferritin | ng/mL | 355 | 292 | 178 | 124 | 1.2E−07 |
| Glutathione S-Transferase | ng/mL | 22 | 25 | 0.62 | 0.55 | 1.5E−07 |
| Prostate Specific Antigen, Free | ng/mL | 0.47 | 0.35 | 0.20 | 0.24 | 3.5E−07 |
| IL-3 | ng/mL | 0.46 | 0.28 | 0.087 | 0.059 | 8.2E−07 |
| Tissue Factor | ng/mL | 5.4 | 3.6 | 2.9 | 2.1 | 2.0E−06 |
| Alpha-Fetoprotein | ng/mL | 7.5 | 3.7 | 4.7 | 2.7 | 3.6E−06 |
| Prostatic Acid Phosphatase | ng/mL | 0.41 | 0.30 | 0.24 | 0.15 | 6.4E−06 |
| Stem Cell Factor | pg/mL | 98 | 54 | 44 | 37 | 3.3E−05 |
| MIP-1beta | pg/mL | 147 | 159 | 79 | 52 | 7.5E−05 |
| Carcinoembryonic Antigen | ng/mL | 3.5 | 4.4 | 1.7 | 1.3 | 1.3E−04 |
| IL-13 | pg/mL | 57 | 35 | 41 | 14 | 1.9E−04 |
| TNF-alpha | pg/mL | 17 | 27 | 7.3 | 5.7 | 6.3E−04 |
| IgE | ng/mL | 260 | 327 | 108 | 161 | 1.4E−03 |
| Fatty Acid Binding Protein | ng/mL | 20 | 26 | 6.6 | 6.7 | 1.5E−03 |
| ENA-78 | ng/mL | 1.2 | 1.3 | 0.64 | 0.70 | 1.9E−03 |
| IL-1beta | pg/mL | 7.0 | 6.9 | 4.0 | 3.1 | 2.2E−03 |
| Brain-Derived Neurotrophic | ng/mL | 3.6 | 4.7 | 2.2 | 1.8 | 3.2E−03 |
| Factor | | | | | | |
| Apolipoprotein A1 | mg/mL | 0.68 | 0.48 | 0.84 | 0.21 | 4.0E−03 |
| Serum Amyloid P | ug/mL | 34 | 7.0 | 30 | 8.7 | 5.0E−03 |
| Growth Hormone | ng/mL | 1.5 | 1.5 | 0.72 | 1.4 | 5.2E−03 |
| Beta-2 Microglobulin | ug/mL | 2.3 | 0.98 | 2.0 | 0.55 | 5.7E−03 |
| Lipoprotein (a) | ug/mL | 99 | 112 | 52 | 84 | 7.5E−03 |
| MMP-9 | ng/mL | 217 | 159 | 313 | 235 | 9.3E−03 |
| Thyroid Stimulating Hormone | uIU/mL | 2.1 | 1.4 | 1.5 | 1.1 | 1.0E−02 |
| Alpha-2 Macroglobulin | mg/mL | 0.39 | 0.65 | 0.23 | 0.078 | 1.0E−02 |
| Complement 3 | mg/mL | 1.4 | 0.65 | 1.2 | 0.26 | 1.2E−02 |
| IL-7 | pg/mL | 37 | 22 | 44 | 16 | 1.9E−02 |
| Leptin | ng/mL | 18 | 30 | 11 | 10 | 2.5E−02 |
| IL-6 | pg/mL | 54 | 43 | 30 | 18 | 3.1E−02 |

Analyte levels can be measured using an immunoassay such as an ELISA or a multiplexed method as described below, and in more detail by Chandler et al., U.S. Pat. No. 5,981,180 (Luminex Corporation).

MMP-3 levels above about 1 ng/mL were identified in unstable angina patients and myocardial infarction patients. In contrast, diagnostic sensitivities of cardiac-specific troponins and C-reactive protein in unstable angina were low. In published studies only 22% of patients had a positive result for troponin T, 36% had a positive result for troponin I, and 65% had raised C-reactive protein levels. See, Hamm et al., N. Engl. J. Med., 1997, 337:1648-1653 and Liuzzo et al., N. Engl. J. Med., 1994, 331:417-424. Both biomarkers, nonetheless, are associated with unfavorable outcomes when elevated. Thus, MMP-3 is a valuable unstable plaque biomarker even when troponins and C-reactive protein are not elevated, potentially identifying high-risk patients who otherwise might remain undiagnosed. Without being bound by a particular mechanism, MMP-3 may be directly involved in the pathophysiology of acute coronary syndromes.

SGOT levels below 10 μg/mL were identified in unstable angina patients and myocardial infarction patients. The role SGOT plays in the pathophysiology of acute coronary syndromes is not known.

The analytes used in the method of the invention can be detected, for example, by a binding assay. For example, a sandwich immunoassay can be performed by capturing MMP-3 and SGOT from a biological sample with antibodies having specific binding affinity for each protein, which then can be detected with a labeled antibody having specific binding affinity for each analyte. Alternatively, standard immunohistochemical techniques can be used to detect MMP-3 and SGOT using such antibodies. Antibodies having affinity for MMP-3 and SGOT are available.

The term “binding reagent” and like terms, refers to any compound, composition or molecule capable of specifically or substantially specifically (that is with limited cross-reactivity) binding another compound or molecule, which, in the case of immune-recognition is an epitope. The binding reagents typically are antibodies, preferably monoclonal antibodies, or derivatives or analogs thereof, but also include, without limitation: Fᵥ fragments; single chain Fᵥ (scFᵥ) fragments; Fab′ fragments; F(ab′)₂ fragments; humanized antibodies and antibody fragments; camelized antibodies and antibody fragments; and multivalent versions of the foregoing. Multivalent binding reagents also may be used, as appropriate, including without limitation: monospecific or bispecific antibodies, such as disulfide stabilized Fᵥ fragments, scFᵥ tandems ((scFᵥ)₂ fragments), diabodies, tribodies or tetrabodies, which typically are covalently linked or otherwise stabilized (i.e., leucine zipper or helix stabilized) scFᵥ fragments. “Binding reagents” also include aptamers, as are described in the art.

Methods of making antigen-specific binding reagents, including antibodies and their derivatives and analogs and aptamers, are well-known in the art. Polyclonal antibodies can be generated by immunization of an animal. Monoclonal antibodies can be prepared according to standard (hybridoma) methodology. Antibody derivatives and analogs, including humanized antibodies can be prepared recombinantly by isolating a DNA fragment from DNA encoding a monoclonal antibody and subcloning the appropriate V regions into an appropriate expression vector according to standard methods. Phage display and aptamer technology is described in the literature and permit in vitro clonal amplification of antigen-specific binding reagents with very affinity low cross-reactivity. Phage display reagents and systems are available commercially, and include the Recombinant Phage Antibody System (RPAS), commercially available from Amersham Pharmacia Biotech, Inc. of Piscataway, N.J. and the pSKAN Phagemid Display System, commercially available from MoBiTec, LLC of Marco Island, Fla. Aptamer technology is described for example and without limitation in U.S. Pat. Nos. 5,270,163, 5,475096, 5,840,867 and 6,544,776.

The ELISA and Luminex LabMAP immunoassays described below are examples of sandwich assays. The term “sandwich assay” refers to an immunoassay where the antigen is sandwiched between two binding reagents, which are typically antibodies. The first binding reagent/antibody being attached to a surface and the second binding reagent/antibody comprising a detectable group. Examples of detectable groups include, for example and without limitation: fluorochromes, enzymes, epitopes for binding a second binding reagent (for example, when the second binding reagent/antibody is a mouse antibody, which is detected by a fluorescently-labeled anti-mouse antibody), for example an antigen or a member of a binding pair, such as biotin. The surface may be a planar surface, such as in the case of a typical grid-type array (for example, but without limitation, 96-well plates and planar microarrays), as described herein, or a non-planar surface, as with coated bead array technologies, where each “species” of bead is labeled with, for example, a fluorochrome (such as the Luminex technology described herein and in U.S. Pat. Nos. 6,599,331, 6,592,822 and 6,268,222), or quantum dot technology (for example, as described in U.S. Pat. No. 6,306,610).

In the bead-type immunoassays described in the examples below, the Luminex LabMAP system is utilized. The LabMAP system incorporates polystyrene microspheres that are dyed internally with two spectrally distinct fluorochromes. Using precise ratios of these fluorochromes, an array is created consisting of 100 different microsphere sets with specific spectral addresses. Each microsphere set can possess a different reactant on its surface. Because microsphere sets can be distinguished by their spectral addresses, they can be combined, allowing up to 100 different analytes to be measured simultaneously in a single reaction vessel. A third fluorochrome coupled to a reporter molecule quantifies the biomolecular interaction that has occurred at the microsphere surface. Microspheres are interrogated individually in a rapidly flowing fluid stream as they pass by two separate lasers in the Luminex analyzer. High-speed digital signal processing classifies the microsphere based on its spectral address and quantifies the reaction on the surface in a few seconds per sample.

For the assays described herein, the bead-type immunoassays are preferable for a number of reasons. As compared to ELISAs, costs and throughput are far superior. As compared to typical planar antibody microarray technology (for example, in the nature of the BD Clontech Antibody arrays, commercially available form BD Biosciences Clontech of Palo Alto, Calif.), the beads are far superior for quantitation purposes because the bead technology does not require pre-processing or titering of the plasma or serum sample, with its inherent difficulties in reproducibility, cost and technician time. For this reason, although other immunoassays, such as, without limitation, ELISA, RIA and antibody microarray technologies, are capable of use in the context of the present invention, but they are not preferred. As used herein, “immunoassays” refer to immune assays, typically, but not exclusively sandwich assays, capable of detecting and quantifying a desired blood biomarker, namely at least one of MMP-3, SGOT, IL-18, Factor VII, ICAM-1, Creatine Kinase-MB, MCP-1, Myoglobin, C Reactive Protein, TIMP-1, Ferritin and Glutathione S-Transferase, or any combination of the foregoing.

Data generated from an assay to determine blood levels of one, two, three, or four or more of the biomarkers MMP-3, SGOT, IL-18, Factor VII, ICAM-1, Creatine Kinase-MB, MCP-1, Myoglobin, C Reactive Protein, von Willebrand Factor, TIMP-1, Ferritin, Glutathione S-Transferase, Prostate Specific Antigen (free), IL-3, Tissue Factor, alpha-Fetoprotein, Prostatic Acid Phosphatase, Stem Cell Factor, MIP-1-beta, Carcinoembryonic Antigen, IL-13, TNF-alpha, IgE, Fatty Acid Binding Protein, ENA-78, IL-1-beta, Brain-Derived Nerotrophic Factor, Apolipoprotein A1, Serum Amyloid P, Growth Hormone, Beta-2 microglobulin, Lipoprotein (a), MMP-9, Thyroid Stimulating hormone, alpha-2 Macroglobulin, Complement 3, IL-7, Leptin and IL-6 can be used to determine the likelihood of a patient suffering a heart attack. As shown herein, if any one or more, two or more, typically three or four or more of the following conditions are met in a patient's blood, MMP-3 (about 1 ng/mL or above), SGOT (about 10 μg/mL or below), IL-18 (about 300 pg/mL or above), Factor VII (about 320 ng/mL or above), ICAM-1 (about 170 ng/mL or above), Creatine Kinase-MB (about 5 ng/mL or above), MCP-1 (about 275 pg/mL or above), Myoglobin (about 30 ng/mL or above), C Reactive Protein (about 11 g/mL or above), TIMP-1 (about 120 ng/mL or above), Ferritin (about 300 ng/mL or above), or Glutathione S-Transferase (about 2 ng/mL or above), there is a very high likelihood that the patient has suffered or is suffering from a heart attack. In one embodiment, either an elevated MMP-3 level or a depressed SGOT level alone, relative to the level of the biomarker of interest in a population of normal or control patients, indicates the existence of ACS in the patient with about a 97-99% level of certainty. (See, Table 2, discussed further elsewhere herein.)

In the context of the present disclosure, “blood” includes any blood fraction, for example serum, that can be analyzed according to the methods described herein. Serum is a standard blood fraction that can be tested, and is tested in the Examples below. By measuring blood levels of a particular biomarker, it is meant that any appropriate blood fraction can be tested to determine blood levels and that data can be reported as a value present in that fraction. As a non-limiting example, the blood levels of a biomarker can be presented as 50 pg/mL serum.

As described above, methods for diagnosing ACS by determining levels of specific identified blood biomarkers are provided. Also provided are methods of detecting preclinical ACS comprising determining the presence and/or velocity of specific identified biomarkers in a patient's blood. By velocity it is meant the changes in the concentration of the biomarker in a patient's blood over time. Longitudinal data has value in determining the velocity of specific biomarkers in a patient's blood for predicting the onset of clinical ACS. Biomarkers with demonstrable velocity indicative of preclinical ACS include: MMP-3, IL-18, Factor VII, ICAM-1, Creatine Kinase-MB, MCP-1, Myoglobin, C Reactive Protein, von Willebrand Factor, TIMP-1, Ferritin, Glutathione S-Transferase, Prostate Specific Antigen (free), IL-3, Tissue Factor, alpha-Fetoprotein, Prostatic Acid Phosphatase, Stem Cell Factor, MIP-1-beta, Carcinoembryonic Antigen, IL-13, TNF-alpha, IgE, Fatty Acid Binding Protein, ENA-78, IL-1-beta, Brain-Derived Nerotrophic Factor, Apolipoprotein A1, Serum Amyloid P, Growth Hormone, Beta-2 microglobulin, Lipoprotein (a), MMP-9, Thyroid Stimulating hormone, alpha-2 Macroglobulin, Complement 3, IL-7, Leptin and IL-6 which increase in concentration beginning at about 1-24 months prior to clinical onset of ACS; and SGOT which decreases in concentration beginning at 1-24 months prior to clinical onset of ACS.

### Example I

Patient Population. The patient population was chosen based on an elevated level of CKMB and Troponin. Both of these enzymes were followed for each patient over time at a hospital until a conclusive diagnosis of ACS was made. The sample of blood, which was tested, was obtained on admission to the hospital. The normal or control patient population was chosen from a wellness clinic. These control patients had no indication of suffering from cardiovascular disease. Consent and blood specimens from all participants were obtained under IRB Protocol.

Collection and storage of blood specimens: Ten mL of peripheral blood was drawn from subjects using standardized phlebotomy procedures. Blood samples were collected without anticoagulant into two 5 mL red top vacutainers, sera were separated by centrifugation, and all specimens were immediately frozen and stored in the dedicated −80 C freezer. All blood samples were logged on the study computer to track information such as storage date, freeze/thaw cycles and distribution.

Development of Luminex assay. The reagents for multiplex system were developed using antibody pairs purchased from R&D Systems (Minneapolis, Minn.), Fitzgerald Industries International (Concord, Mass.) or produced by well known immunological methods. Capture antibodies were monoclonal and detection antibodies were polyclonal. Capture Abs were covalently coupled to carboxylated polystyrene microspheres number 74 purchased from Luminex Corporation (Austin, Tex.). Covalent coupling of the capture antibodies to the microspheres was performed by following the procedures recommended by Luminex. In short, the microspheres' stock solutions were dispersed in a sonification bath (Sonicor Instrument Corporation, Copiaque, N.Y.) for 2 min. An aliquot of 2.5×10⁶ microspheres was resuspended in microtiter tubes containing 0.1 M sodium phosphate buffer, pH 6.1 (phosphate buffer), to a final volume of 80 μL. This suspension was sonicated until a homogeneous distribution of the microspheres was observed. Solutions of N-hydroxy-sulfosuccinimide (Sulfo-NHS) and 1-ethyl-3-(3-dimethylaminopropyl)-carbodiimide hydrochloride (Pierce), both at 50 mg/mL, were prepared in phosphate buffer, and 10 μL of each solution was sequentially added to stabilize the reaction and activate the microspheres. This suspension was incubated for 10 min at room temperature and then resuspended in 250 μL of PBS containing 50 μg of antibody. The mixture was incubated overnight in the dark with continuous shaking. Microspheres were then incubated with 250 μL of PBS-0.05% Tween 20 for 4 h. After aspiration, the beads were blocked with 1 mL of PBS-1% BSA-0.1% sodium azide. The microspheres were counted with a hemacytometer and stored at a final concentration of 10⁶ microspheres per mL in the dark at 4 C. Coupling efficiency of monoclonal antibodies was tested by staining 2,000 microspheres with PE-conjugated goat anti-mouse IgG (BD Biosciences, San Diego, Calif.). Detection Abs were biotinylated using EZ-Link Sulfo-NHS-Biotinylation Kit (Pierce, Rockford, Ill.) according to manufacturer's protocol. The extent of biotin incorporation was determined using HABA assay and was 20 moles of biotin per mole of protein. The assays were further optimized for concentration of detection Ab and for incubation times. Sensitivity of the newly developed assays were determined using serially diluted purified proteins. Intra-assay variability, expressed as a coefficient of variation, was calculated based on the average for patient samples and measured twice at two different time points. The intra-assay variability within the replicates is expressed as an average coefficient of variation. Inter-assay variability was evaluated by testing quadruplicates of each standard and sample with an average of 16.5% (data not shown). Newly developed kits were multiplexed together and the absence of cross-reactivity was confirmed according to Luminex protocol.

Examples of some commercial sources of matched antibody cytokine pairs include MAB636 EGF (R&D Systems, Minneapolis, Minn.), BAF236 G-CSF (R&D Systems), DY214 IL-6 (R&D Systems), DY206 IL-8 (R&D Systems), DY208 IL-12p40 (R&D Systems), DY1240 MCP-1 (R&D Systems), DY279 VEGF (R&D Systems), DY293 CA-125 (M002201, M002203, Fitzgerald Industries International, Inc., Concord, Mass.).

Additionally, CA-125 reagent for multiplex system was developed using antibody pair purchased from Fitzgerald Industries International (Concord, Mass.). Capture antibody was monoclonal and detection antibody was sheep polyclonal. Capture Ab was biotinylated using EZ-Link Sulfo-NHS-Biotinylation Kit (Pierce, Rockford, Ill.) according to the manufacturer's protocol. The extent of biotin incorporation was determined using HABA assay and was 20 moles of biotin per mole of protein. Capture Ab was covalently coupled to carboxylated polystyrene microspheres number 74 purchased from Luminex Corporation (Austin, Tex.). Covalent coupling of the capture antibodies to the microspheres was performed by following the procedures recommended by Luminex. In short, the microspheres' stock solutions were dispersed in a sonification bath (Sonicor Instrument Corporation, Copiaque, N.Y.) for 2 min. An aliquot of 2.5×10⁶ microspheres was resuspended in microtiter tubes containing 0.1 M sodium phosphate buffer, pH 6.1 (phosphate buffer), to a final volume of 80 μL. This suspension was sonicated until a homogeneous distribution of the microspheres was observed. Solutions of N-hydroxy-sulfosuccinimide (Sulfo-NHS) and 1-ethyl-3-(3-dimethylaminopropyl)-carbodiimide hydrochloride (Pierce), both at 50 mg/mL, were prepared in phosphate buffer, and 10 μL of each solution was sequentially added to stabilize the reaction and activate the microspheres. This suspension was incubated for 10 min at room temperature and then resuspended in 250 μL of PBS containing 50 μg of antibody. The mixture was incubated overnight in the dark with continuous shaking. Microspheres were then incubated with 250 μL of PBS-0.05% Tween 20 for 4 h. After aspiration, the beads were blocked with 1 mL of PBS-1% BSA-0.1% sodium azide. The microspheres were counted with a hemacytometer and stored at a final concentration of 10⁶ microspheres per mL in the dark at 4 C. Coupling efficiency of monoclonal antibodies was tested by staining 2,000 microspheres with PE-conjugated goat anti-mouse IgG (BD Biosciences, San Diego, Calif.). The assay was further optimized for concentration of detection Ab and for incubation times. Sensitivity of the newly developed assay as determined in a Luminex assay using serially diluted purified CA-125, was 20 IU. Intra-assay variability, expressed as a coefficient of variation, was calculated based on the average for patient samples and measured twice at least two different time points. The intra-assay variability within the replicates presented as an average coefficient of variation was 8.5%. Interassay variability was evaluated by testing quadruplicates of each standard and 10 samples. The variabilities of these samples were between 10 and 22%, with an average of 16.5%. Next, the anti-CA-125 microspheres were combined with the existing multiplex kit.

Statistical Analysis of Data. All statistical analyses were conducted using S-Plus statistical software (Seattle, Wash.: Math Soft, Inc., 1999). The data were first randomly split into a training and test set; described in Table C. Logistic regression (Hosmer, D W, S Lemeshow, Applied Logistic Regression. New York, N.Y.: John Wiley & Sons, 1989) was then used to calculate the optimal weighting of each biomarker and the subsequent predicted probability of being a case. All predicted probabilities ≧0.5 were categorized as a predicted case; predicted probabilities <0.5 were categorized as a predicted control. After fitting a logistic model to the training set, classification of disease status was then calculated for the test set.

### Results

Serum concentrations of biomarkers by LabMap technology. Circulating concentrations of different serum biomarkers were evaluated in a multiplexed assay using LabMap technology in blood of patients from ACS and control groups. Table 1 lists the analytes that are statistically different between the two groups.

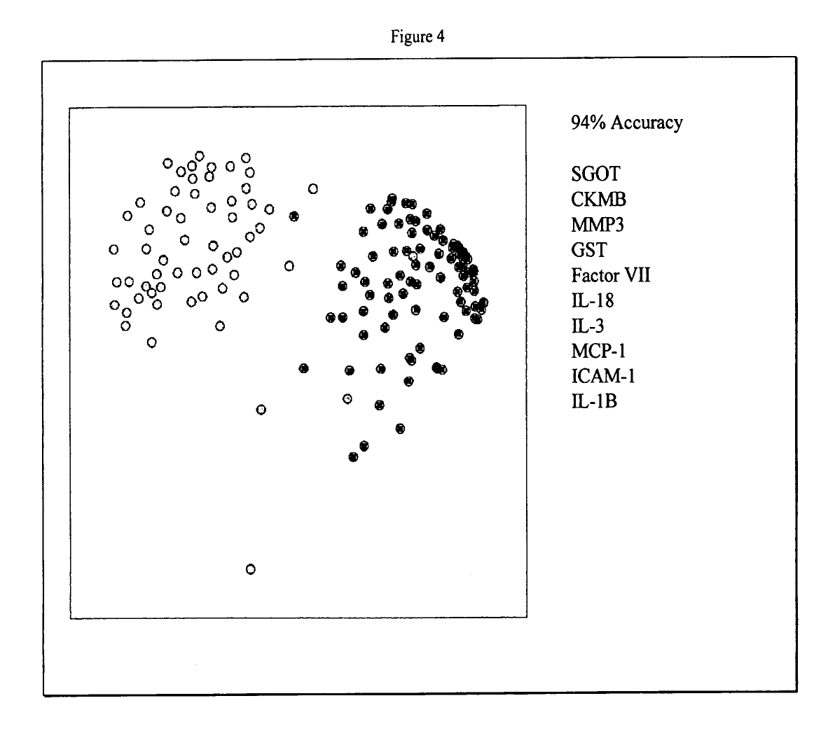

Table 2 illustrates the diagnostic accuracy obtained by testing for each individual analyte and determining how useful it would be as a diagnostic tool. In the case of CKMB, which was part of our selection criteria, there would have been 3 people out of 89 that would have been admitted with the expectation that they were having an ACS event but in fact they were normal (i.e., three false positives). Also 6 people would have been sent home when they were actually having a heart attack (i.e., six false negatives). If we only used the results from MMP-3 there would have only been 2 people that would have been diagnosed incorrectly compared to 9 people with CKMB. Thus, we have discovered two new biomarkers that are more accurate on entrance to the hospital that CKMB, the current standard. The best individual biomarkers would appear to be MMP-3 and SGOT. The best combination of biomarkers would appear to be the twelve analytes that are shown in FIG. 4.

TABLE 2
Analyte Accuracy for ACS

| | Neg | Pos | Accuracy |
|---|---|---|---|
| MMP-3 | | | |
| True Neg | 89 | 0 | |
| True Pos | 2 | 57 | 99% |
| SGOT | | | |
| True Neg | 88 | 1 | |
| True Pos | 3 | 56 | 97% |
| CKMB | | | |
| True Neg | 86 | 3 | |
| True Pos | 6 | 53 | 94% |
| GST | | | |
| True Neg | 85 | 4 | |
| True Pos | 8 | 51 | 92% |
| Factor VII | | | |
| True Neg | 84 | 5 | |
| True Pos | 8 | 51 | 91% |
| ICAM-1 | | | |
| True Neg | 82 | 7 | |
| True Pos | 9 | 50 | 89% |
| MCP-1 | | | |
| True Neg | 84 | 5 | |
| True Pos | 13 | 46 | 88% |
| IL-18 | | | |
| True Neg | 82 | 7 | |
| True Pos | 12 | 47 | 87% |
| Myoglobin | | | |
| True Neg | 80 | 9 | |
| True Pos | 16 | 43 | 83% |
| CRP | | | |
| True Neg | 78 | 11 | |
| True Pos | 21 | 38 | 78% |
| Ferritin | | | |
| True Neg | 79 | 10 | |
| True Pos | 25 | 34 | 76% |
| TIMP-1 | | | |
| True Neg | 80 | 9 | |
| True Pos | 27 | 32 | 76% |

Proximity Map Analysis. The proximity map data analysis is conducted with a software program that groups samples by their similarities in analyte concentration patterns. A unique chemical signature is generated using the concentration of the analytes measured in each sample. The relationship of each sample signature is visualized in the Galaxy™ projection. The Galaxy™ is a proximity map, such that the closer two objects are in the visualization, the closer their chemical signatures are, and thus the more similar they are to one another. The axes are dimensionless (a result of being derived from a principal component analysis), and thus the visualization is not a typical X-Y scatter plot in which moving along one axis means increasing or decreasing a single value. The two axes of the Galaxy™ are defined by the first two principal components, a common method to reduce complex data. The placement of objects (record points) is done using a set of heuristics that have been designed to maximize the preservation of spatial relationships that existed in the high-dimensional space of the original data while minimizing the overlap that can occur when doing simple projections.

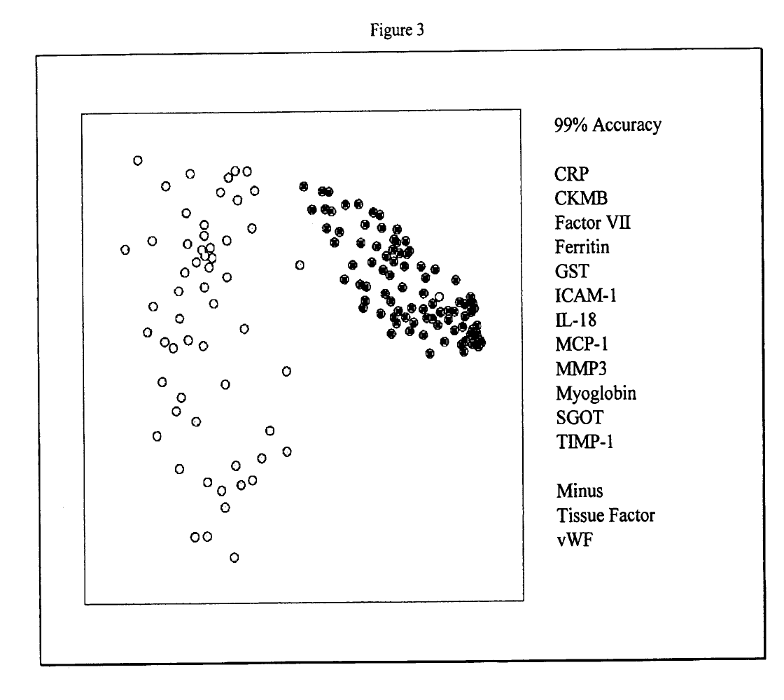

An examination of the FIGs, shows that the red circles (light gray, the ACS patients) are separated from the blue circles (dark gray, controls) to various degrees with all of the plots attaining fairly good separation. FIG. 3 provides what is possibly the best separation. If an unknown sample is tested for the analytes listed in FIG. 3, then the location of the patient (from whom the unknown sample is taken) on the plot would be indicative of whether that patient is having a heart attack or not. The space between the two clusters would appear to be an indeterminate area. In FIG. 3 there appears to be one ACS patient that lands in the middle of the control population. This results might be a false negative or the original clinical diagnosis might have been simply incorrect.

Rates of classification accuracy (in discriminating controls from ACS patients) were then obtained using 10-fold cross-validation and a Receiver Operating Characteristic (ROC) curve was generated. The sensitivity and specificity of the method depend on the cut-point (i.e. predicted probability from the classification tree) used to classify each subject as either a case or control. Using the standard cut-point of 0.5 (i.e. everyone with a predicted probability above 0.5 is classified as a cancer case) gives 100% sensitivity, 86% specificity, and 93% correctly classified. Fixing the specificity at 91% still leads to a very high sensitivity, at 95.5% (again with 93% correctly classified). Alternatively, a specificity of 95.3% corresponds to a sensitivity of 84.1% (and 90.0% correctly classified). The total area under the receiver operating characteristic (ROC) curve was near one (which would represent perfect classification), at 0.966.

### Example II

### Development of LabMAP Assays for Circulating Antibodies

Assays were performed in filter-bottom 96-well microplates (Millipore). Purified antigens of interest were coupled to Luminex beads as described for antibodies. Antigen-coupled beads were pre-incubated with blocking buffer containing 4% BSA for 1 h at room temperature on microtiter shaker. Beads were then washed three times with washing buffer (PBS, 1% BSA, 0.05% Tween 20) using a vacuum manifold followed by incubation with 50 μL blood serum diluted 1:250 for 30 min at 4 C. This dilution was selected as an optimal for recovery of anti-IL-18 IgG based on previous serum titration (data not shown). Next, washing procedure was repeated as above and beads were incubated with 50 μL/well of 4 μg/mL PE-conjugated antibody raised against human IgG (Jackson Laboratories), for 45 min in the dark with the constant shaking. Wells were washed twice, assay buffer was added to each well and samples were analyzed using the Bio-Plex suspension array system (Bio-Rad Laboratories, Hercules, Calif.). For standard curve, antigen-coupled beads were incubated with serially diluted human antibodies against specific antigens. Purification of monospecific human antibodies is described above. Data analysis was performed using five-parametric-curve fitting.

In another particular aspect of the invention, the expression profiles of one or a plurality of the disclosed markers could provide valuable molecular tools for examining the molecular basis of drug responsiveness in ACS and for evaluating the efficacy of drugs for ACS or their side effects. Changes in the expression profile from a baseline profile while the cells are exposed to various modifying conditions, such as contact with a drug or other active molecules can be used as an indication of such effects.

Therefore, the invention provides a test for use in determining whether a patient suffering from ACS will respond to therapy comprising the steps of, performing the diagnostic steps of the inventive method described hereinabove for body samples obtained respectively from an individual treated for ACS with a pharmaceutically acceptable agent and an individual not diagnosed with ACS, and determining the responsiveness to drug therapy.

Monitoring the influence of agents (e.g., drug compounds) on the level of expression of a marker of the invention can be advantageously applied in clinical trials. For example, the effectiveness of an agent to affect marker expression can be monitored in clinical trials of subjects receiving treatment for ACS. In a preferred embodiment, the present invention provides a method for monitoring the efficacy of treatment of a subject with an agent (e.g., an agonist, antagonist, peptidomimetic, protein, peptide, nucleic acid, small molecule, or other drug candidate) comprising the steps of: (i) obtaining a pre-administration sample from a subject prior to administration of the agent; (ii) detecting the level of expression of one or more selected markers of the invention in the pre-administration sample; (iii) obtaining one or more post-administration samples from the subject; (iv) detecting the level of expression of the marker (s) in the post-administration samples; (v) comparing the level of expression of the marker (s) in the pre-administration sample with the level of expression of the marker (s) in the post-administration sample or samples; and (vi) altering the administration of the agent to the subject accordingly.

For example, modified administration of the agent can be desirable to increase expression of the novel diagnostic marker(s) to higher levels than detected, i.e., to increase the effectiveness of the agent. Alternatively, increased/decreased administration of the agent can be desirable to increase/decrease the effectiveness of the agent, respectively.

As used herein, the term “candidate agent” or “drug candidate” can be natural or synthetic molecules such as proteins or fragments thereof, antibodies, small molecule inhibitors or agonists, nucleic acid molecules, e.g., antisense nucleotides, ribozymes, double-stranded RNAs, organic and inorganic compounds and the like.

In another particular aspect of the present invention, a method is provided for both prophylactic and therapeutic methods of treating a subject having, or at risk of having, a heart attack (or ACS). Administration of a prophylactic agent can occur prior to the manifestation of symptoms characteristic of the heart attack, such that development of the ACS is prevented or delayed in its progression.

Examples of suitable therapeutic agents include, but are not limited to, antisense nucleotides, ribozymes, double-stranded RNAs, ligands, small molecules and antagonists as described more in detail below.

In a particular embodiment, the invention provides a method for treating or preventing ACS in an individual comprising the step of administering to said individual a therapeutically effective amount of a modulating compound that modulates expression or activity of one or more of the genes, gene expression, or protein products of the group of genes MMP-3 and/or SGOT, so that at least one symptom of ACS is ameliorated.

In another aspect, the invention provides a method for treating or preventing ACS in an individual comprising the step of administering to said individual a therapeutically effective amount of a modulating compound that modulates expression or activity of one or more of the genes or gene expression products of the group of genes IL-18, Factor VII, ICAM-1, Creatine Kinase-MB, MCP-1, Myoglobin, C Reactive Protein, TIMP-1, Ferritin, and/or Glutathione S-Transferase, so that at least one symptom of ACS is ameliorated.

In another particular aspect of the invention, by virtue of the differential expression of the inventive diagnostic markers, it is possible to utilize these markers to enhance the certainty of prediction of whether a particular drug treatment in a patient will be effective in treating ACS. Therefore, the invention provides a method for identifying candidate agents for use in the treatment of ACS comprising the steps of: a) contacting a sample of tissue, preferably cardiac tissue, subject to toxicity with a candidate agent; b) determining from the tissue the level of gene or protein expression corresponding to one or more genes selected among MMP-3 and/or SGOT, to obtain a first set of value; and c) comparing the first set of value with a second set of value corresponding to the level of gene expression, assessed for the same gene (s) and under identical condition as for step b) in a tissue subject to toxicity not induced by the candidate agent, wherein a first value substantially equal or greater than the second value for MMP-3 and/or SGOT expression is an indication that the candidate agent is ameliorating ACS symptoms. Similarly, a first value substantially equal or lower than the second value for at least one of IL-18, Factor VII, ICAM-1, Creatine Kinase-MB, MCP-1, Myoglobin, C Reactive Protein, TIMP-1, Ferritin, or Glutathione S-Transferase expression is an indication that the candidate agent is ameliorating ACS symptoms.

In another particular aspect of the invention, a method is provided for identifying candidate agents for use in the treatment of ACS comprising the steps of (a) contacting a sample of a tissue, preferably cardiac tissue, subject to toxicity with a candidate agent; (b) determining from the tissue the level of gene or protein expression corresponding to one or more genes selected among MMP-3, SGOT, IL-18, Factor VII, ICAM-1, Creatine Kinase-MB, MCP-1, Myoglobin, C Reactive Protein, TIMP-1, Ferritin, or Glutathione S-Transferase, to obtain a first set of value(s); and (c) comparing the first set of values with a second set of values corresponding to the level of expression assessed for the same gene(s) and under identical condition as for step b) in a tissue subject to toxicity not induced by the candidate agent wherein a first value substantially greater than the second value for said gene expression is an indication that the candidate agent is ameliorating ACS symptoms. In a preferred embodiment, only the MMP-3 and/or SGOT genes are used in the assay.

In a preferred embodiment, the means for determining the level of gene expression comprises oligonucleotides specific for a marker gene. Particularly preferred are methods selected from Northern blot analysis, reverse transcription PCR or real time quantitative PCR, branched DNA, nucleic acid sequence based amplification (NASBA), transcription-mediated amplification, ribonuclease protection assay, and microarrays. Sequences of the marker genes disclosed herein are readily available from publicly accessible gene banks, such as GENBANK, and for sake of conciseness, are not detailed here.

Particularly useful methods for detecting the level of mRNA transcripts obtained from the novel markers disclosed herein include hybridization of labeled mRNA to an ordered array of oligonucleotides. Such a method allows the level of transcription of a plurality of these genes to be determined simultaneously to generate gene expression profiles or patterns. The gene expression profile derived from the sample obtained from the subject can, in another embodiment, be compared with the gene expression profile derived form the sample obtained from the disease-free subject, and thereby determine whether the subject has or is at risk of developing ACS.

The gene expressions of the markers can also be preferably assessed in the form of a kit using RT-PCR, a high throughput technology: The well-known technique RT-PCR reaction exploits the 5′ nuclease activity of AmpliTaqGold DNAPolymerase to cleave a TaqMan probe during PCR. The probe consists of an oligonucleotide (usually >20 mer) with a 5′-reporter dye and a 3′-quencher dye. The fluorescent reporter dye, such as FAM(6-carboxyfluorescein), is covalently linked to the 5′ end of the oligonucleotide. The reporter is quenched by TAMRA (6-carboxy-N,N,N′,N′-tetramethylrhodamine) attached via a linker arm that is located at the 3′ end.

Oligonucleotide probes used for each marker should derive from the nucleotide sequence of the gene of such marker, the selection of the appropriate oligonucleotide sequence being now a matter of standard routine technique for one skilled in the art. Again, because the DNA sequences of the respective genes described herein are freely available, they have not be reiterated here.

In addition to the drug screening methods known, cell-free assays can also be used to identify compounds which are capable of interacting with proteins encoded by the markers taught herein (e.g., MMP-3, SGOT), to alter the activity of the protein or its binding partner. Cell-free assays can also be used to identify compounds, which modulate the interaction between the encoded protein and its binding partner such as a target peptide.

In one embodiment, cell-free assays for identifying such compounds comprise a reaction mixture containing a marker protein and a test compound or a library of test compounds in the presence or absence of the binding partner, e.g., a biologically inactive target peptide, or a small molecule. Interaction between molecules can also be assessed by using real-time BIA (Biomolecular Interaction Analysis, Pharmacia Biosensor (AB) which detects surface plasmon resonance, an optical phenomenon. Formation of a complex between the protein and its binding partner can be detected by using detectably labeled proteins such as radiolabeled, fluorescently labeled, or enzymatically labeled protein or its binding partner, by immunoassay or by chromatographic detection.

In another embodiment, activity of a target RNA (preferable mRNA) species, specifically its rate of translation, can be controllably inhibited by the controllable application of antisense nucleic acids. An “antisense” nucleic acid as used herein refers to a nucleic acid capable of hybridizing to a sequence-specific (e.g., non-poly A) portion of the target RNA, for example its translation initiation region, by virtue of some sequence complementarity to a coding and/or non-coding region. The antisense nucleic acids of the invention can be oligonucleotides that are double-stranded or single-stranded, RNA or DNA or a modification or derivative thereof, which can be directly administered in a controllable manner to a cell or which can be produced intracellularly by transcription of exogenous, introduced sequences in controllable quantities sufficient to perturb translation of the target RNA.

Preferably, antisense nucleic acids are of at least six nucleotides and are preferably oligonucleotides (ranging from 6 to about 200 oligonucleotides).

As discussed above, antisense nucleotides can be delivered to cells, which express the described genes in vivo by various techniques, e.g., injection directly into cardiac tissue, entrapping the antisense nucleotide in a liposome, by administering modified antisense nucleotides which are targeted to the heart cells by linking the antisense nucleotides to peptides or antibodies that specifically bind receptors or antigens expressed on the cell surface.

However, with the above-mentioned delivery methods, it may be difficult to attain intracellular concentrations sufficient to inhibit translation of endogenous mRNA. Accordingly, in an alternative embodiment, the nucleic acid comprising an antisense nucleotide sequence is placed under the transcriptional control of a promoter, i.e., a DNA sequence which is required to initiate transcription of the specific genes, to form an expression construct. The antisense nucleic acids of the invention are controllably expressed intracellularly by transcription from an exogenous sequence. If the expression is controlled to be at a high level, a saturating perturbation or modification results. In conclusion, antisense nucleic acids can be routinely designed to target virtually any mRNA sequence including the marker genes cited in the present document, and a cell can be routinely transformed with or exposed to nucleic acids coding for such antisense sequences such that an effective and controllable or saturating amount of the antisense nucleic acid is expressed. Hence, the translation of virtually any RNA species in a cell can be modified or perturbed.

In addition, the activities of marker proteins can be modified or perturbed in a controlled or a saturating manner by exposure to exogenous drugs or ligands. Since the methods of this invention are often applied to testing or confirming the usefulness of various drugs to treat cardiac disorders, drug exposure is an important method of modifying/perturbing cellular constituents, both mRNA's and expressed proteins.

In a preferable case, a drug is known that interacts with only one marker protein in the cell and alters the activity of only that one marker protein, either increasing or decreasing the activity. Graded exposure of a cell to varying amounts of that drug thereby causes graded perturbations of network models having that marker protein as an input. Saturating exposure causes saturating modification/perturbation.

The term “antagonist” refers to a molecule which, when bound to the protein encoded by the gene, inhibits its activity. Antagonists can include, but are not limited to, peptides, proteins, carbohydrates, and small molecules. In a particularly useful embodiment, the antagonist is an antibody specific for the markers disclosed (e.g., MMP-3 and/or SGOT). The antibody alone may act as an effector of therapy or it may recruit other cells to actually effect cell killing.

In the case of treatment with an antisense nucleotide, the method comprises administering a therapeutically effective amount of an isolated nucleic acid molecule comprising an antisense nucleotide sequence derived from at least one marker identified hereinabove wherein the antisense nucleotide has the ability to change the transcription/translation of the at least one gene. In the case of treatment with an antagonist, the method comprises administering to a subject a therapeutically effective amount of an antagonist that inhibits or activates a protein encoded by at least one marker identified above.

A “therapeutically effective amount” of an isolated nucleic acid molecule comprising an antisense nucleotide, nucleotide sequence encoding a ribozyme, double-stranded RNA, or antagonist, refers to a sufficient amount of one of these therapeutic agents to treat ACS. The determination of a therapeutically effective amount is well within the capability of those skilled in the art. For any therapeutic, the therapeutically effective dose can be estimated initially either in cell culture assays, e.g., of neoplastic cells, or in animal models, usually rats, mice, rabbits, dogs or pigs. The animal model may also be used to determine the appropriate concentration range and route of administration. Such information can then be used to determine useful doses and routes for administration in humans.

Therapeutic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio between toxic and therapeutically effects is the therapeutic index, and it can be expressed as the ratio LD50/ED50. Antisense nucleotides, ribozymes, double-stranded RNAs and antagonists that exhibit large therapeutic indices are preferred. The data obtained from cell culture assays and animal studies is used in formulating a range of dosage for human use. The dosage contained in such compositions is preferably within a range of circulating concentrations that include the ED50 with little or no toxicity. The dosage varies within this range, depending upon the dosage form employed, sensitivity of the patient, and the route of administration.

The exact dosage may be determined by the practitioner, in light of factors related to the subject that requires treatment. Dosage and administration are adjusted to provide sufficient levels of the active moiety or to maintain the desired effect. Factors that may be taken into account include the severity of the disease state, general health of the subject, age, weight and gender of the subject, diet, time and frequency of administration, drug combination(s), reaction sensitivities, and tolerance/response to therapy.

Normal dosage amounts may vary form 0.1 to 100,000 micrograms, up to a total dosage of about 1 g, depending upon the route of administration. Guidance as to particular dosages and methods of delivery is provided in the literature and generally available to practitioners in the art. Those skilled in the art will employ different formulations for nucleotides than for antagonists.

For therapeutic applications, the antisense nucleotides, nucleotide sequences encoding ribozymes, double-stranded RNAs (whether entrapped in a liposome or contained in a viral vector) and antibodies are preferably administered as pharmaceutical compositions containing the therapeutic agent in combination with one or more pharmaceutically acceptable carriers. The compositions may be administered alone or in combination with at least one other agent, such as stabilizing compound, which may be administered in any sterile, biocompatible pharmaceutical carrier, including, but not limited to, saline, buffered saline, dextrose and water. The compositions may be administered to a patient alone or in combination with other agents, drugs or hormones.

The pharmaceutical compositions may be administered by a number of routes including, but not limited to, oral, intravenous, intramuscular, intra-articular, intra-arterial, intramedullary, intrathecal, intraventricular, transdermal, subcutaneous, intraperitoneal, intranasal, enteral, topical, sublingual, or rectal means. In addition to the active ingredient, these pharmaceutical compositions may contain suitable pharmaceutically acceptable carriers comprising excipients and auxiliaries which facilitate processing of the active compounds into preparations which can be used pharmaceutical.

Pharmaceutical compositions for oral administration can be formulated using pharmaceutical acceptable carriers well known in the art in dosages suitable for oral administration. Such carriers enable the pharmaceutical compositions to be formulated as tablets, pills, capsules, liquids, gels, syrups, slurries, suspensions, and the like, for ingestion by the patient.

Whereas particular embodiments of the invention have been described herein for the purpose of illustrating the invention and not for the purpose of limiting the same, it will be appreciated by those of ordinary skill in the art that numerous variations of the details, materials and arrangement of parts may be made within the principle and scope of the invention without departing from the invention as described in the appended claims.

What is claimed is:

1. A method of diagnosing acute coronary syndrome (ACS) in a human subject suspected of suffering from ACS, comprising:
(a) obtaining a fluid sample from a human subject suspected of suffering from ACS;
(b) determining the concentration of MMP-3 in said fluid sample;
(c) deciding if the determined concentration of MMP-3 in said fluid sample is statistically different from that found in a control group of human subjects, whereby a statistically different elevated concentration of MMP-3 supports a positive diagnosis of ACS.

2. The method of claim 1 in which said human subject is complaining of chest pains.

3. The method of claim 1 in which said fluid sample is selected from the group consisting of whole blood, plasma, serum, or urine.

4. The method of claim 1 in which a determined concentration of about 1 ng/mL or above of MMP-3 in said fluid sample supports a positive diagnosis.

5. A method of diagnosing acute coronary syndrome (ACS) in a human subject suspected of suffering from ACS, comprising:
(a) obtaining a fluid sample from a human subject suspected of suffering from ACS;
(b) determining the concentration of SGOT in said fluid sample;
(c) deciding if the determined concentration of SGOT in said fluid sample is statistically different from that found in a control group of human subjects,
whereby a statistically different depressed concentration of SGOT supports a positive diagnosis of ACS.

6. The method of claim 5 in which said human subject is complaining of chest pains.

7. The method of claim 5 in which said fluid sample is selected from the group consisting of whole blood, plasma, serum, or urine.

8. The method of claim 5 in which a determined concentration of about 10 μg/mL or below of SGOT in said fluid sample supports a positive diagnosis.

9. A method of diagnosing acute coronary syndrome (ACS) in a human subject suspected of suffering from ACS, comprising:
(a) obtaining a fluid sample from a human subject suspected of suffering from ACS;
(b) determining the concentrations of MMP-3 and SGOT in said fluid sample;
(c) deciding if the determined concentrations of MMP-3 and SGOT in said fluid sample are statistically different from that found in a control group of human subjects,
whereby a statistically different elevated concentration of MMP-3 and a statistically different depressed concentration of SGOT support a positive diagnosis of ACS.

10. The method of claim 9 which further comprises determining the concentration in said fluid sample of at least one of IL-18, Factor VII, ICAM-1, Creatine Kinase-MB, MCP-1, Myoglobin, C Reactive Protein, TIMP-1, Ferritin, or Glutathione S-Transferase, or any combination thereof.

11. The method of claim 10 in which statistically different elevated concentrations, compared to control levels, of all analytes except SGOT support a positive diagnosis of ACS.

12. The method of claim 9 in which concentrations are determined by conducting one or more immunoassays.

13. The method of claim 9 which further comprises determining the concentration in said fluid sample of at least one of IL-18, Factor VII, ICAM-1, Creatine Kinase-MB, MCP-1, Myoglobin, C Reactive Protein, von Willebrand Factor, TIMP-1, Ferritin, Glutathione S-Transferase, Prostate Specific Antigen (free), IL-3, Tissue Factor, alpha-Fetoprotein, Prostatic Acid Phosphatase, Stem Cell Factor, MIP-1-beta, Carcinoembryonic Antigen, IL-13, TNF-alpha, IgE, Fatty Acid Binding Protein, ENA-78, IL-1-beta, Brain-Derived Nerotrophic Factor, Apolipoprotein A1, Serum Amyloid P, Growth Hormone, Beta-2 microglobulin, Lipoprotein (a), MMP-9, Thyroid Stimulating hormone, alpha-2 Macroglobulin, Complement 3, IL-7, Leptin, or IL-6, or any combination thereof.

14. The method of claim 13 in which statistically different elevated concentrations, compared to control levels, of all analytes except SGOT support a positive diagnosis of ACS.

15. The method of claim 9 in which a subject's determined concentrations of analytes in said fluid sample are presented in a proximity map, whereby the proximity of a subject's determined concentrations to a cluster of other subjects' determined concentrations, who were previously diagnosed as having suffered from ACS, contributes to a positive diagnosis of ACS.

16. A kit comprising reagents for determining the concentration in a fluid sample of a panel of analytes including MMP-3, SGOT and one or more of IL-18, Factor VII, ICAM-1, Creatine Kinase-MB, MCP-1, Myoglobin, C Reactive Protein, TIMP-1, Ferritin, or Glutathione S-Transferase.

17. The kit of claim 16 which includes antibodies against a panel of analytes including MMP-3, SGOT, IL-18, Factor VII, ICAM-1, Creatine Kinase-MB, MCP-1, Myoglobin, C Reactive Protein, TIMP-1, Ferritin, and Glutathione S-Transferase.

18. The kit of claim 16 which includes reagents immobilized on a substrate.

19. The kit of claim 18 which the substrate comprises a two-dimensional array, a microtiter plate, or multiple bead sets.

20. The method of claim 9 which includes applying a statistical method selected from the group consisting of linear regression analysis, classification tree analysis and heuristic nave Bayes analysis.

21. The method of claim 10 in which determined concentrations of one or more of the following analytes in said fluid sample supports a positive diagnosis of ACS: IL-18 (about 300 pg/mL or above), Factor VII (about 320 ng/mL or above), ICAM-1 (about 170 ng/mL or above), Creatine Kinase-MB (about 5 ng/mL or above), MCP-1 (about 275 pg/mL or above), Myoglobin (about 30 ng/mL or above), C Reactive Protein (about 11 μg/mL or above), TIMP-1 (about 120 ng/mL or above), Ferritin (about 300 ng/mL or above), or Glutathione S-Transferase (about 2 ng/mL or above), or any combination thereof.
